(19) United States
(12) Patent Application Publication
(10) Pub. No.: US 2004/0115167 A1
(43) Pub. Date: Jun. 17, 2004

(54) Drug delivery device and method having coated microprojections incorporating vasoconstrictors

(76) Inventors: Michel Cormier, Mountain View, CA (US); James Matriano, Mountain View, CA (US); WeiQi Lin, Palo Alto, CA (US); Wendy Young, Cupertino, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/674,626

(22) Filed: Sep. 29, 2003

Related U.S. Application Data
(60) Provisional application No. 60/415,121, filed on Sep. 30, 2002.

(51) Int. Cl. A61K 38/16; A61K 38/21; A61K 9/70; A61M 31/00

(52) U.S. Cl. 424/85.1; 424/449; 424/85.2; 424/85.4; 514/12; 514/649; 514/8; 604/500

(57) ABSTRACT

A device and method are provided for percutaneous transdermal delivery of a biologically active agent. The coating formulation containing the biologically active agent and a vasoconstrictor is applied to the skin piercing elements using known coating techniques and then dried. The device is applied to the skin of a living animal, causing the microprojections to pierce the stratum corneum and to deliver an effective dose of the biologically active agent and vasoconstrictor to the animal.

### FIELD OF THE INVENTION

This application claims the benefit of U.S. Provisional Application No. 60/415,121, filed Sep. 30, 2002.

This invention relates to administering and enhancing transdermal delivery of a biologically active agent across the skin. More particularly, the invention relates to a percutaneous delivery system for administering a biologically active agent through the stratum corneum using skin piercing microprojections that have a dry coating of the biologically active agent and a vasoconstrictor. Transdermal delivery of the agent is facilitated when the microprojections pierce the skin of a patient and the patient's interstitial fluid contacts and dissolves the biologically active agent and the vasoconstrictor.

### BACKGROUND OF THE INVENTION

Drugs are most conventionally administered either orally or by injection. Unfortunately, many drugs are completely ineffective or have radically reduced efficacy when orally administered since they either are not absorbed or are adversely affected before entering the bloodstream and thus do not possess the desired activity. On the other hand, the direct injection of the drug into the bloodstream, while assuring no modification of the drug during administration, is a difficult, inconvenient, painful and uncomfortable procedure which sometimes results in poor patient compliance.

Hence, in principle, transdermal delivery provides for a method of administering drugs that would otherwise need to be delivered via hypodermic injection or intravenous infusion. Transdermal drug delivery offers improvements in both of these areas. Transdermal delivery when compared to oral delivery avoids the harsh environment of the digestive tract, bypasses gastrointestinal drug metabolism, reduces first-pass effects, and avoids the possible deactivation by digestive and liver enzymes. Conversely, the digestive tract is not subjected to the drug during transdermal administration. Indeed, many drugs such as aspirin have an adverse effect on the digestive tract. However, in many instances, the rate of delivery or flux of many agents via the passive transdermal route is too limited to be therapeutically effective.

Skin is not only a physical barrier that shields the body from external hazards, but is also an integral part of the immune system. The immune function of the skin arises from a collection of residential cellular and humoral constituents of the viable epidermis and dermis with both innate and acquired immune functions, collectively known as the skin immune system.

One of the most important components of the skin immune system are the Langerhan's cells (LC) which are specialized antigen presenting cells found in the viable epidermis. LC's form a semi-continuous network in the viable epidermis due to the extensive branching of their dendrites between the surrounding cells. The normal function of the LC's is to detect, capture and present antigens to evoke an immune response to invading pathogens. LC's perform his function by internalizing epicutaneous antigens, trafficking to regional skin-draining lymph nodes, and presenting processed antigens to T cells.

The effectiveness of the skin immune system is responsible for the success and safety of vaccination strategies that have been targeted to the skin. Vaccination with a live-attenuated smallpox vaccine by skin scarification has successfully led to global eradication of the deadly small pox disease. Intradermal injection using ⅕ to 1/10 of the standard IM doses of various vaccines has been effective in inducing immune responses with a number of vaccines while a low-dose rabies vaccine has been commercially licensed for intradermal application.

As an alternative, transdermal delivery provides for a method of administering vaccines that would otherwise need to be delivered via hypodermic injection, intravenous infusion or orally. Transdermal vaccine delivery offers improvements in both of these areas. Transdermal delivery when compared to oral delivery avoids the harsh environment of the digestive tract, bypasses gastrointestinal drug metabolism, reduces first-pass effects, and avoids the possible deactivation by digestive and liver enzymes. Conversely, the digestive tract is not subjected to the vaccine during transdermal administration. However, in many instances, the rate of delivery or flux of many vaccines via the traditional passive transdermal route is too limited to be immunologically effective.

The word “transdermal” is used herein as a generic term referring to passage of an agent across the skin layers. The word “transdermal” refers to delivery of an agent (e.g., a therapeutic agent such as a drug or an immunologically active agent such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources including electricity (e.g., iontophoresis) and ultrasound (e.g., phonophoresis). While drugs do diffuse across both the stratum corneum and the epidermis, the rate of diffusion through the stratum corneum is often the limiting step. Many compounds, in order to achieve an effective dose, require higher delivery rates than can be achieved by simple passive transdermal diffusion. When compared to injections, transdermal agent delivery eliminates the associated pain and reduces the possibility of infection.

Theoretically, the transdermal route of administration could be advantageous for the delivery of many therapeutic proteins, because proteins are susceptible to gastrointestinal degradation and exhibit poor gastrointestinal uptake and transdermal devices are more acceptable to patients than injections. However, the transdermal flux of medically useful peptides and proteins is often insufficient to be therapeutically effective due to the relatively large size/molecular weight of these molecules. Often the delivery rate or flux is insufficient to produce the desired effect or the agent is degraded prior to reaching the target site, for example while in the patient's bloodstream.

Transdermal drug delivery systems generally rely on passive diffusion to administer the drug while active transdermal drug delivery systems rely on an external energy source (e.g., electricity) to deliver the drug. Passive transdermal drug delivery systems are more common. Passive transdermal systems have a drug reservoir containing a high concentration of drug. The reservoir is adapted to contact the skin which enables the drug to diffuse through the skin and into the body tissues or bloodstream of a patient. The transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (keratinocytes) surrounded by lipid bilayers. This highly-ordered structure of the lipid bilayers confers a relatively impermeable character to the stratum corneum.

One common method of increasing the passive transdermal diffusional drug flux involves pre-treating the skin with, or co-delivering with the drug, a skin permeation enhancer. A permeation enhancer, when applied to a body surface through which the drug is delivered, enhances the flux of the drug therethrough. However, the efficacy of these methods in enhancing transdermal protein flux has been limited, at least for the larger proteins, due to their size.

Active transport systems use an external energy source to assist drug flux through the stratum corneum. One such enhancement for transdermal drug delivery is referred to as “electrotransport.” This mechanism uses an electrical potential, which results in the application of electric current to aid in the transport of the agent through a body surface, such as skin. Other active transport systems use ultrasound (phonophoresis) and heat as the external energy source.

There also have been many attempts to mechanically penetrate or disrupt the outermost skin layers thereby creating pathways into the skin in order to enhance the amount of agent being transdermally delivered. Early vaccination devices known as scarifiers generally had a plurality of tines or needles which were applied to the skin to and scratch or make small cuts in the area of application. The vaccine was applied either topically on the skin, such as U.S. Pat. No. 5,487,726 issued to Rabenau or as a wetted liquid applied to the scarifier tines such as U.S. Pat. No. 4,453,926 issued to Galy, or U.S. Pat. No. 4,109,655 issued to Chacornac, or U.S. Pat. No. 3,136,314 issued to Kravitz. Scarifiers have been suggested for intradermal vaccine delivery in part because only very small amounts of the vaccine need to be delivered into the skin to be effective in immunizing the patient. Further, the amount of vaccine delivered is not particularly critical since an excess amount also achieves satisfactory immunization.

However, a serious disadvantage in using a scarifier to deliver a drug is the difficulty in determining the transdermal drug flux and the resulting dosage delivered. Also, due to the elastic, deforming and resilient nature of skin to deflect and resist puncturing, the tiny piercing elements often do not uniformly penetrate the skin and/or are wiped free of a liquid coating of an agent upon skin penetration.

Additionally, due to the self healing process of the skin, the punctures or slits made in the skin tend to close up after removal of the piercing elements from the stratum corneum. Thus, the elastic nature of the skin acts to remove the active agent liquid coating that has been applied to the tiny piercing elements upon penetration of these elements into the skin. Furthermore the tiny slits formed by the piercing elements heal quickly after removal of the device, thus limiting the passage of the liquid agent solution through the passageways created by the piercing elements and in turn limiting the transdermal flux of such devices.

Other devices which use tiny skin piercing elements to enhance transdermal drug delivery are disclosed in European Patent EP 0 407063A1, U.S. Pat. No. 5,879,326 issued to Godshall, et al., U.S. Pat. No. 3,814,097 issued to Ganderton, et al., U.S. Pat. No. 5,279,544 issued to Gross, et al., U.S. Pat. No. 5,250,023 issued to Lee, et al., U.S. Pat. No. 3,964,482 issued to Gerstel, et al., Reissue 25,637 issued to Kravitz, et al., and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated by reference in their entirety. These devices use piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

Generally, these systems include a reservoir for holding the drug and also a delivery system to transfer the drug from the reservoir through the stratum corneum, such as by hollow tines of the device itself. One example of such a device is disclosed in WO 93/17754, which has a liquid drug reservoir. The reservoir must be pressurized to force the liquid drug through the tiny tubular elements and into the skin. Disadvantages of devices such as these include the added complication and expense for adding a pressurizable liquid reservoir and complications due to the presence of a pressure-driven delivery system.

Instead of a physical reservoir, it is possible to have the drug that is to be delivered coated the microprojections, as disclosed in U.S. patent application Ser. No. 10/045,842, which is fully incorporated by reference herein. This eliminates the necessity of a separate physical reservoir and developing a drug formulation or composition specifically for the reservoir.

Vasoconstrictors are well known pharmacological agents that are being used in therapeutics to reduce the peripheral blood flow. Vasoconstrictors are used chiefly to decrease conjunctival congestion, to decrease nasal secretions, and in the case of simultaneous injection of a vasoconstrictor with local anesthetics, to retard the absorption of the anesthetic and increase the duration of the anesthesia. Most compounds possessing vasoconstrictive activity are thought to exert their action through alpha-adrenergic action. Stimulation of the alpha-adrenergic receptors results in vasoconstriction in the precapillary vessels of skin or mucosa.

The efficiency of delivery of a biologically active agent from coated microprojections is at least partially dependent upon the length of the microprojections. The greater the length of the microprojection, the greater the physical area of the microprojection that can be coated with drug or vaccine. In addition, the longer the projection, the larger the area of coated projection that can be inserted sufficiently into the stratum corneum. Thus, the larger the area of coated microprojection that will be exposed to interstitial fluid. This will increase the amount of the drug or vaccine that is dissolved.

However, the greater the length of the microprojections, the larger and deeper will be the slits that are created in the skin when the microprojections are applied to the skin. This can increase the amount of bleeding at the application site. Bleeding is not only aesthetically displeasing and uncomfortable for the patient but is also a biohazard risk to ancillary health care workers and others working or living with the patient. In addition, excessive bleeding can result in the flushing out of the biologically active agent from the application site.

If the projections are long enough, the biologically active agent can be inserted into the underlying capillary bed resulting in systemic exposure to the biologically active agent. This is a desirable feature when administering drugs. Microprojection length must be balanced with the bleeding that will occur if the microprojection length is too great.

Bleeding has been a limiting factor in the development of microprojection arrays as an effective transdermal delivery platform. Bleeding is a particular problem for patients who are hemophiliacs or for those patients taking anti-coagulants including but not limited to such over-the-counter products as aspirin. The present invention overcomes this limitation and allows the use of longer microprojections which would otherwise cause unacceptable bleeding.

In particular with regard to vaccines, by decreasing the amount of vaccine that is exposed to capillary bed, a greater amount find its way to the lymphatic system which will increase the probably of immunogenic response by the patient to the vaccine. Decreasing capillary blood flow would increase the exposure the vaccine to the lymphatic system.

Another complication of traditional vaccine delivery is the possibility of anaphylactic shock occurring at a later time. Anaphylaxis is the local or systemic allergenic reaction which may occur when an antigen is re-introduced after a time lapse. Introduction of the vaccine during booster shots can cause anaphylactic shock if the body is subjected to the vaccine too quickly. If the vaccine is in any manner injected into the systemic circulation, the patient is then at greater risk for an anaphylactic reaction.

Thus, there is a need to deliver biologically active agents at an effective rate, via application of coated microprojection arrays, while at the same time minimizing bleeding and blood flow from the site of application and reducing exposure of the biologically active agents to systemic circulation for the purpose of effecting a controlled release of drugs to the circulation, increasing the immunogenic response to vaccines, and reducing the possibility of inducing anaphylactic shock by a rapid repeat exposure to a vaccine. In addition, there is a need for a device and method to deliver biologically active agents to patients who are hemophiliacs and to those patients taking anti-coagulants, including, but not limited to, such over-the-counter products as aspirin.

It is therefore an object of the present invention to provide a transdermal drug delivery apparatus having coated microprotrusions and a method for employing same that substantially reduces or eliminates the aforementioned drawbacks and disadvantages associated with prior art drug delivery systems.

It is another object of the present invention to provide a transdermal drug delivery apparatus that includes microprotrusions coated with an active or drug and a vasoconstrictor.

It is another object of the present invention to provide a transdermal drug delivery apparatus having a coated microprojection array that delivers biological active agents at an effective rate.

It is another object of the present invention to provide a transdermal drug delivery apparatus and method for delivering a biologically active agent and vasoconstrictor through the stratum corneum of a patient via a plurality of coated stratus corneum-piercing microprojections.

It is yet another object of the present invention to provide an effective method of delivering biological active agents that (i) minimizes bleeding and blood flow from the site of application, (ii) reduces exposure of the biological active agents to systemic circulation for the purpose of effecting a controlled release of the agents, (iii) increases the immunogenic response to vaccines, and (iv) reduces the possibility of inducing anaphylactic shock by a rapid repeat exposure to a vaccine.

### SUMMARY OF THE INVENTION

In accordance with the above objects and those that will be mentioned and will become apparent below, the present invention comprises a device and method for delivering a biologically active agent and vasoconstrictor through the stratum corneum of preferably a mammal and, most preferably, a human, by having a coating on a plurality of stratum corneum-piercing microprojections.

The present invention is further directed to a device and method for delivering a biologically active agent and vasoconstrictor through the stratum corneum of a human patient having hemophilia and those taking anti-coagulants, including, but not limited to, such over-the-counter products as aspirin, by limiting bleeding from the slits formed by the microprojections by having the coating on a plurality of stratum corneum-piercing microprojections that contains, in addition to the biologically active agent, a biologically effective amount of a vasoconstrictor.

A preferred embodiment of this invention consists of a device for delivering through the stratum corneum, a biologically active agent which has been coated on a plurality of microprojections by applying to the microprojections a solution of the biologically active agent and a vasoconstrictor, which is then dried to form the coating. Optionally, the microprojections are surface treated to enhance the uniformity of the coating that is formed on the microprojections.

The device comprises a member having a plurality, and preferably a multiplicity, of stratum corneum-piercing microprojections. In a preferred embodiment, each of the microprojections has a length of less than 1000 microns, or, if longer than 1000 microns, then means are provided to ensure that the microprojections penetrate the skin to a depth of no more than 1000 microns.

Each microprojection includes a dry coating preferably having a thickness of less than 50 microns adhered thereon. The coating, before drying, comprises a solution of a biologically active agent and a vasoconstrictor. The solution, once coated onto the surfaces of the microprojections, provides a biologically effective amount of the biologically active agent and a biologically effective amount of the vasoconstrictor. The coating is further dried onto the microprojections using drying methods known in the art.

Another preferred embodiment of this invention consists of a method of making a device for transdermally delivering a biologically active agent. The method comprises providing a member having a plurality of stratum corneum-piercing microprojections. A solution of the biologically active agent plus a vasoconstrictor is applied to the microprojections and then dried to form a dry agent- and vasoconstrictor-containing coating thereon. The biologically active agent is sufficiently potent to be biologically effective in a dose that can be contained within the coatings. The vasoconstrictor is also sufficiently potent to exert is local vasoconstrictive effect at doses than can be contained in the coating. The composition can be prepared at any temperature as long as the biologically active agent is not rendered inactive due to the conditions. The solution, once coated onto the surfaces of the microprojections, provides a biologically effective amount of the biologically active agent and the vasoconstrictor.

The coating thickness is preferably less than the thickness of the microprojections, more preferably, the thickness is less than 50 microns and, most preferably, less than 25 microns. Generally, the coating thickness is an average thickness measured over the coated microprojection area.

Preferred biologically active agents include ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserelin, triptorelin, interferon alpha, interferon beta, interferon gamma, FSH, EPO, GM-CSF, G-CSF, IL-10, glucagon, growth hormone releasing factor (GRF) and analogs of these agents including pharmaceutically acceptable salts thereof. Preferred biologically active agents further include conventional vaccines, recombinant protein vaccines, DNA vaccines and therapeutic cancer vaccines.

Though DNA vaccines are generally considered to be a pharmacological agent, they are discussed herein with the vaccines because of their similar ability to affect an immunological response.

The vasoconstrictors can comprise any number of compounds, including, but not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the like.

Preferred vasoconstrictors include epinephrine, naphazoline, tetrahydrozoline indanazoline, metizoline, tramazoline, tymazoline, oxymetazoline and xylometazoline. The preferred concentration of the vasoconstrictor is 0.1 wt. % to 10 wt. % of the coating.

The coating can be applied to the microprojections using known coating methods. For example, the microprojections can be immersed or partially immersed into an aqueous coating solution of the agent as described in pending U.S. patent application Ser. No. 10/099,604, filed Mar. 15, 2002. Alternatively, the coating solution can be sprayed onto the microprojections. Preferably, the spray has a droplet size of about 10-200 picoliters. More preferably, the droplet size and placement is precisely controlled using printing techniques so that the coating solution is deposited directly onto the microprojections and not onto other “non-piercing” portions of the member having the microprojections.

In another aspect of the invention, the stratum corneum-piercing microprojections are formed from a sheet wherein the microprojections are formed by etching or punching the sheet and then the microprojections are folded or bent out of a plane of the sheet. While the biologically active agent coating can be applied to the sheet before formation of the microprojections, preferably the coating is applied after the microprojections are cut or etched out but prior to being folded out of the plane of the sheet. More preferred is that the coating is applied after the microprojections have been folded or bent out from the plane of the sheet.

### BRIEF DESCRIPTION OF THE FIGURES

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection array;

FIG. 2 is a perspective view of the microprojection array of FIG. 1 with a coating deposited onto the microprojections;

FIG. 3 is a graph showing the effect of a vasoconstrictor on bleeding at the microprojection application site;

FIG. 4 is a graph showing blood flow at the application site of a microprojection array which had a coating containing a vasoconstrictor; and

FIG. 5 is a graph showing normalized blood flow at the application site of a microprojection array which a coating containing a vasoconstrictor.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only, and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a vasoconstrictor” includes two or more such vasoconstrictors and the like.

### Definitions

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more biologically active agents may be coated onto the microprojections resulting in co-delivery of the biologically active agents.

The term “biologically active agent”, as used herein, refers to a composition of matter or mixture containing a drug which is pharmacologically effective when administered in a therapeutically effective amount. Examples of such active agents include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotropin (ACTH), ACTH analogs such as ACTH (1-24), calcitonin, parathyroid hormone (PTH), vasopressin, deamino [Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10) and glucagon. It is to be understood that more than one agent may be incorporated into the agent formulation in the method of this invention, and that the use of the term “active agent” in no way excludes the use of two or more such agents or drugs. The agents can be in various forms, such as free bases, acids, charged or uncharged molecules, components of molecular complexes or nonirritating, pharmacologically acceptable salts. Also, simple derivatives of the agents (such as ethers, esters, amides, etc) which are easily hydrolyzed at body pH, enzymes, etc., can be employed.

The term “biologically active agent”, as used herein, also refers to a composition of matter or mixture containing a vaccine or other immunologically active agent or an agent which is capable of triggering the production of an immunologically active agent, and which is directly or indirectly immunologically effective when administered in an immunologically effective amount.

The term “vaccine”, as used herein, refers to conventional and/or commercially available vaccines, including, but not limited to, flu vaccines, Lyme disease vaccine, rabies vaccine, measles vaccine, mumps vaccine, chicken pox vaccine, small pox vaccine, hepatitis vaccine, pertussis vaccine, and diphtheria vaccine, recombinant protein vaccines, DNA vaccines and therapeutic cancer vaccines. The term “vaccine” thus includes, without limitation, antigens in the form of proteins, polysaccharides, oligosaccharides, lipoproteins, weakened or killed viruses such as cytomegalovirus, hepatitis B virus, hepatitis C virus, human papillomavirus, rubella virus, and varicella zoster, weakened or killed bacteria such as bordetella pertussis, clostridium tetani, corynebacterium diphtheriae, group A streptococcus, legionella pneumophila, neisseria meningitides, pseudomonas aeruginosa, streptococcus pneumoniae, treponema pallidum, and vibrio cholerae and mixtures thereof.

The term “biologically effective amount” or “biologically effective rate” shall be used when the biologically active agent is a pharmaceutically active agent and refers to the amount or rate of the pharmacologically active agent needed to effect the desired therapeutic, often beneficial, result. The amount of agent employed in the coatings will be that amount necessary to deliver a therapeutically effective amount of the agent to achieve the desired therapeutic result. In practice, this will vary widely depending upon the particular pharmacologically active agent being delivered, the site of delivery, the severity of the condition being treated, the desired therapeutic effect and the dissolution and release kinetics for delivery of the agent from the coating into skin tissues. It is not practical to define a precise range for the therapeutically effective amount of the pharmacologically active agent incorporated into the microprojections and delivered transdermally according to the methods described herein.

The term “biologically effective amount” or “biologically effective rate” shall also be used when the biologically active agent is an immunologically active agent and refers to the amount or rate of the immunologically active agent needed to stimulate or initiate the desired immunologic, often beneficial result. The amount of the immunologically active agent employed in the coatings will be that amount necessary to deliver an amount of the agent needed to achieve the desired immunological result. In practice, this will vary widely depending upon the particular immunologically active agent being delivered, the site of delivery, and the dissolution and release kinetics for delivery of the agent from the coating into skin tissues.

The term “vasoconstrictor”, as used herein, refers to a composition of matter or mixture that narrows the lumen of blood vessels and, hence, reduces peripheral blood flow. Examples of suitable vasoconstrictors include, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human. The piercing elements should not pierce the skin to a depth which causes bleeding.

In one embodiment of the invention, the piercing elements have a projection length less than 1000 microns. In a further embodiment, the piercing elements have a projection length of less than 500 microns, more preferably, less than 250 microns. The microprojections typically have a width and thickness of about 5 to 50 microns. The microprojections may be formed in different shapes, such as needles, hollow needles, blades, pins, punches, and combinations thereof.

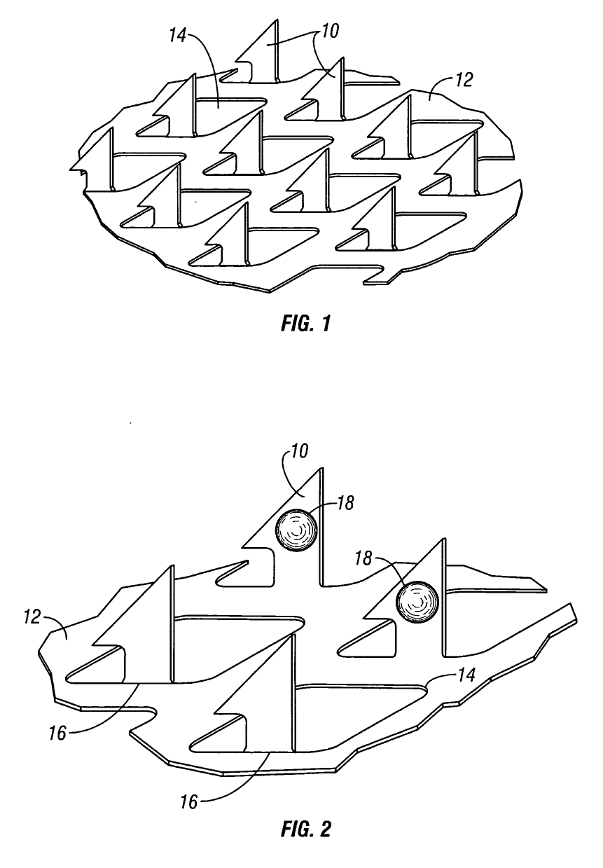

The term “microprojection array”, as used herein, refers to a plurality of microprojections arranged in an array for piercing the stratum corneum. The microprojection array may be formed by etching or punching a plurality of microprojections from a thin sheet and folding or bending the microprojections out of the plane of the sheet to form a configuration such as that shown in FIG. 1. The microprojection array may also be formed in other known manners, such as by forming one or more strips having microprojections along an edge of each of the strip(s) as disclosed in Zuck, U.S. Pat. No. 6,050,988. The microprojection array may include hollow needles which hold a dry pharmacologically active agent.

References to the area of the sheet or member and reference to some property per area of the sheet or member are referring to the area bounded by the outer circumference or border of the sheet.

The term “solution” shall include not only compositions of fully dissolved components but also suspensions of components including, but not limited to, protein virus particles, inactive viruses, and split-virions.

The term “pattern coating”, as used herein, refers to coating an agent onto selected areas of the microprojections. More than one agent may be pattern coated onto a single microprojection array. Pattern coatings can be applied to the microprojections using known micro-fluid dispensing techniques such as micropipeting and ink jet coating.

As indicated above, the present invention provides a device for transdermally delivering a biologically active agent to a patient in need thereof. The device has a plurality of stratum corneum-piercing microprojections extending therefrom. The microprojections are adapted to pierce through the stratum corneum into the underlying epidermis layer, or epidermis and dermis layers. The microprojections have a dry coating thereon which contains the biologically active agent and a vasoconstrictor. Upon piercing the stratum corneum layer of the skin, the agent-containing coating is dissolved by body fluid (intracellular fluids and extracellular fluids such as interstitial fluid) and released into the skin for local or systemic therapy. The vasoconstrictor is also released into the skin when the coating is dissolved, resulting in the inhibition of bleeding and a decrease in blood flow at the site of application of the transdermal device.

The kinetics of the agent-containing coating dissolution and release will depend on many factors including the nature of the biologically active agent, the coating process, the coating thickness and the coating composition (e.g., the presence of coating formulation additives). Depending on the release kinetics profile, it may be necessary to maintain the coated microprojections in piercing relation with the skin for extended periods of time (e.g., up to about 8 hours). This can be accomplished by anchoring the microprojection member to the skin using adhesives or by using anchored microprojections such as described in WO 97/48440, incorporated by reference in its entirety.

FIG. 1 illustrates one embodiment of a stratum corneum-piercing microprojection member for use with the present invention. FIG. 1 shows a portion of the member having a plurality of microprojections 10. The microprojections 10 extend at substantially a 90° angle from sheet 12 having openings 14. Sheet 12 may be incorporated into a delivery patch, including a backing for sheet 12, and may additionally include adhesive for adhering the patch to the skin. In this embodiment, the microprojections are formed by etching or punching a plurality of microprojections 10 from a thin metal sheet 12 and bending microprojections 10 out of the plane of the sheet. Metals such as stainless steel and titanium are preferred. Metal microprojection members are disclosed in Trautman, et al., U.S. Pat. No. 6,083,196; Zuck U.S. Pat. No. 6,050,988; and Daddona, et al., U.S. Pat. No. 6,091,975; the disclosures of which are incorporated herein by reference. Other microprojection members that can be used with the present invention are formed by etching silicon using silicon chip etching techniques or by molding plastic using etched micro-molds. Silicon and plastic microprojection members are disclosed in Godshall, et al., U.S. Pat. No. 5,879,326, the disclosures of which are incorporated herein by reference.

FIG. 2 illustrates the microprojection member having microprojections 10 having a coating 16 which contains the biologically active agent and vasoconstrictor. Coating 16 may partially or completely cover the microprojection 10. For example, the coating can be in a dry pattern coating on the microprojections. The coatings can be applied before or after the microprojections are formed.

The coating on the microprojections can be formed by a variety of known methods. One such method is dip-coating. Dip-coating can be described as a means to coat the microprojections by partially or totally immersing the microprojections into the coating solution. Alternatively the entire device can be immersed into the coating solution. Coating only those portions the microprojection member that pierce the skin is preferred.

By use of the partial immersion technique described above, it is possible to limit the coating to only the tips of the microprojections. There is also a roller coating mechanism that limits the coating to the tips of the microprojection. This technique is described in a United States provisional patent (serial No. 60/276,762), filed 16 Mar. 2001, which is fully incorporated herein by reference.

Other coating methods include spraying the coating solution onto the microprojections. Spraying can encompass formation of an aerosol suspension of the coating composition. In a preferred embodiment an aerosol suspension having a droplet size of about 10 to 200 picoliters is sprayed onto the microprojections and then dried.

In another embodiment, a very small quantity of the coating solution can be deposited onto the microprojections 10, as shown in FIG. 2 as pattern coating 18. The pattern coating 18 can be applied using a dispensing system for positioning the deposited liquid onto the microprojection surface. The quantity of the deposited liquid is preferably in the range of 0.5 to 20 nanoliters/microprojection. Examples of suitable precision-metered liquid dispensers are disclosed in U.S. Pat. Nos. 5,916,524; 5,743,960; 5,741,554; and 5,738,728; the disclosures of which are fully incorporated herein by reference.

Microprojection coating solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

In one embodiment of the invention, the coating solutions used in the present invention are solutions of the biologically active agent and a vasoconstrictor, and, optionally, a wetting agent. In either case, the solution must have a viscosity of less than about 500 centipoise and greater than 3 centipoise in order to effectively coat the microprojection properly.

The desired coating thickness is dependent upon the density of the microprojections per unit area of the sheet and the viscosity and concentration of the coating composition as well as the coating method chosen. Preferably, the coating thickness should be less than 50 microns, more preferably, less than 25 microns, since thicker coatings have a tendency to slough off the microprojections upon stratum corneum piercing. Generally coating thickness is referred to as an average coating thickness measured over the coated microprojection.

In one embodiment, the coating thickness is less than 10 microns as measured from the microprojection surface. More preferably, the coating thickness is in the range of approximately 1 to 10 microns.

The active agent used in the present invention requires that the total amount of agent coated on all of the microprojections of a microprojection array be in the range of 1 microgram to 1 milligram.

Amounts within this range can be coated onto a microprojection array of the type shown in FIG. 1 having the sheet 12 with an area of up to 10 cm² and a microprojection density of up to 1000 microprojections per cm².

As indicated above, the coatings of the invention comprise at least one biologically active agent and at least one vasoconstrictor. Applicants have found that the addition of the vasoconstrictor in the coating facilitates the formation of a depot of the active agent within the skin.

Preferred pharmacologically active agents having the properties described above include, without limitation, desmopressin, luteinizing hormone releasing hormone (LHRH) and LHRH analogs (e.g., goserelin, leuprolide, buserelin, triptorelin), PTH, calcitonin, vasopressin, deamino [Val4, D-Arg8] arginine vasopressin, interferon alpha, interferon beta, interferon gamma, menotropins (urofollotropin (FSH) and leutinizing hormone (LH), erythrepoietrin (EPO), GM-CSF, G-CSF, IL-10, GRF, glucagon, conventional vaccines and DNA vaccines.

Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

Preferred concentration of the vasoconstrictor is in the range of approximately 0.1 wt. % to 10 wt. % of the coating.

In all cases, after a coating has been applied, the coating solution is dried onto the microprojections by various means. In a preferred embodiment the coated device is dried in ambient room conditions. However, various temperatures and humidity levels can be used to dry the coating solution onto the microprojections. Additionally, the devices can be heated, lyophilized, freeze dried or similar techniques used to remove the water from the coating.

Other known formulation adjuvants can be added to the coating solution as long as they do not adversely affect the necessary solubility and viscosity characteristics of the coating solution and the physical integrity of the dried coating.

### EXAMPLES

The following examples are given to enable those skilled in the art to more clearly understand and practice the present invention. They should not be considered as limiting the scope of the invention but merely as being illustrated as representative thereof.

### Example 1

Studies were preformed in which bleeding, produced by the application of a microprojection array, was inhibited by co-delivering the vasoconstrictor epinephrine along with Guinea pig albumin. The Guinea pig albumin was used as a model drug or vaccine. The Guinea pig albumen and epinephrine were dry coated on the microprojections of a microprojection array. A microprojection array having long microprojections (600 microns) were chosen in order to maximize bleeding so that the efficacy of the vasoconstrictor could be more easily evaluated.

An aqueous coating solution containing 200 mg/ml of guinea pig albumin and 50 mg/ml of epinephrine bitartrate was prepared. A control solution was prepared which contained only 200 mg/mL guinea pig albumin in water and no vasoconstrictor. The microprojection arrays that were used a penetration angle of 80°. The penetration angle is defined as the angle between the two upper penetration edges of the microprojection. There were 72 microprojections/cm² and the overall area of the microprojection array was 2 cm².

One group of arrays were dipped into each solution and the excess was wicked off by briefly contacting the microprojection array with tissue. The microprojection arrays were then allowed to air-dry overnight at room temperature.

The systems that were applied comprised a coated microprojection array which was adhered to the center of a low density polyethylene (LDPE) 7 cm² disc, coated with a propriety adhesive. Two systems were applied to each hairless guinea pig. One had been coated with the test solution (albumin and vasoconstrictor) and the other with the control solution (albumin only).

At the time of application, the skin of the flank of the animal was manually stretched bilaterally (opposing forces applied on both sides of the expected application site). System application was performed with an impact applicator which applied a force of 0.4 J. Following application of the systems, the stretching tension was released.

Half of the systems were applied for 5 seconds and then removed. The second half of the systems were applied for 2 minutes and then removed. Therefore there were four test conditions with each condition having been tested with four system.

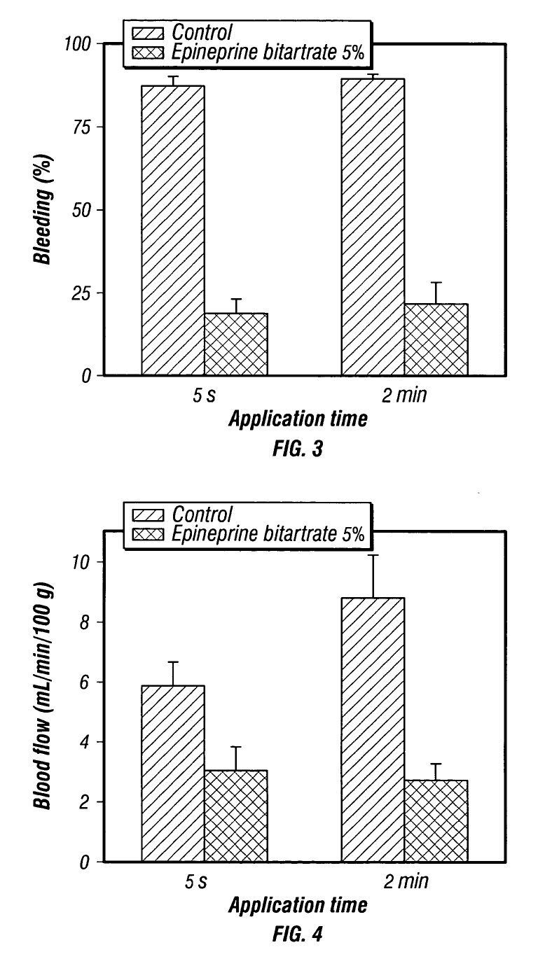

Pictures of the application skin sites were taken 2 minutes after removal of the system. The skin sites were monitored visually for 30 minutes for blanching. Bleeding was evaluated visually from the pictures by estimating the percentage of the microprojection puncture sites that were bleeding. Blood flow (mL/min/100 g) was evaluated with a laser Doppler Velocimeter (LDV) at the skin site immediately prior to application of the system and 2 minutes following removal of the system. For each guinea pig, the blood flow measurement taken prior to system application was subtracted from the second measurement (normalized blood flow). The average of data obtained for each group of four animals were calculated and shown in FIGS. 3-5.

Results demonstrate that co-delivery of the vasoconstrictor epinephrine significantly inhibits bleeding after an application time as short as 5 seconds. As shown in FIG. 3, the percentage of microslits that were found to be bleeding after a 5-second application of a system without epinephrine (control) was about 87%. The percentage of microslits that were bleeding from sites that included epinephrine was 20%. There was little change in the data after a two-minute application of a system.

Blood flow at the application site in animals receiving systems without epinephrine went from 60 ml/min/100 grams to 90 mls/min/100 grams when the application time was extend from 5 seconds to two minutes. However, inclusion of epinephrine resulted in blood flow of 30 mls/min/100 grams for systems that were applied for 5 seconds as well as those systems that were applied for 2 minutes.

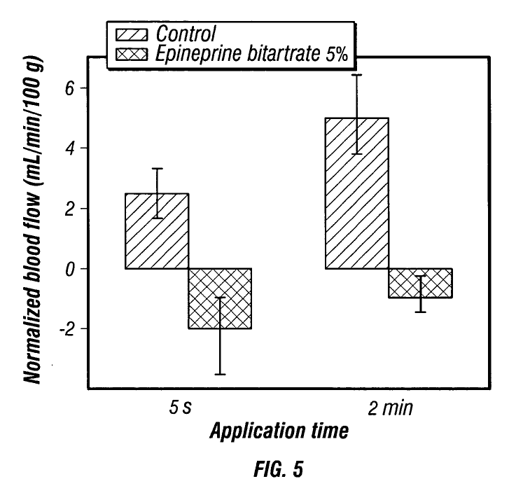

FIG. 5 shows normalized blood flow in the test HGP's. In this graph, data are obtained by subtracting the blood from adjacent control skin. The data shown in this graph demonstrates that epinephrine minimizes the erythema resulting from microprojection array application and even produces some blanching at the application site. The blanching was detectable for about 30 minutes after removal of the microprojection arrays. No blanching at the control sites was observed.

This experiment demonstrates that the use of a microprojection array to co-deliver a vasoconstrictor along with a biologically active agent will result in less bleeding than if no vasoconstrictor is included. In addition, the observed decrease in blood flow in the presence of epinephrine indicates that microprojection array co-delivery of vasoconstrictors with a biologically active agent should prolong agent delivery through formation of a skin depot. Also, microprojection array co-delivery of vasoconstrictors with vaccines should improve the immune response through formation of a skin depot which minimizes systemic exposure. Finally, microprojection array co-delivery of vasoconstrictor with biologically active agents may result in decreased erythema at the site of delivery.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A device for transdermally delivering a biologically active agent, comprising:
a member having a plurality of stratum corneum-piercing microprotrusions; and
a coating disposed on said member, said coating including a biologically active agent and a vasoconstrictor.

2. The device of claim 1, wherein said biologically active agent comprises a vaccine selected from the group consisting of conventional vaccines, recombinant protein vaccines, DNA vaccines and therapeutic cancer vaccines.

3. The device of claim 1, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserlin, triptorelin, interferon alpha, interferon beta, interferon gamma, FSH, EPO, GM-CSF, G-CSF, IL-10, glucagon, growth hormone releasing factor (GRF) and analogs of these agents, including pharmaceutically acceptable salts thereof, and mixtures thereof.

4. The device of claim 1, wherein said vasoconstrictor is selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

5. The device of claim 4, wherein said vasoconstrictor comprises in the range of 0.1-10.0 wt. % of said coating.

6. The device of claim 1, wherein said coating comprises a dry coating, said dry coating comprising an aqueous solution prior to drying.

7. A device for transdermally delivering a biologically active agent and a vasoconstrictor, comprising:
a member having a plurality of stratum corneum-piercing microprotrusions; and
a coating disposed on said member, said coating including a biologically effective amount of a biologically active agent selected from the group consisting of a conventional vaccine, recombinant protein vaccine, DNA vaccine, therapeutic cancer vaccine and mixtures thereof, and a biologically effective amount of a vasoconstrictor selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

8. A device for transdermally delivering a biologically active agent and a vasoconstrictor, comprising:
a member having a plurality of stratum corneum-piercing microprotrusions; and
a coating disposed on said member, said coating including a biologically effective amount of a biologically active agent selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserlin, triptorelin, interferon alpha, interferon beta, interferon gamma, FSH, EPO, GM-CSF, G-CSF, IL-10, glucagon, growth hormone releasing factor (GRF) and analogs thereof, and mixtures thereof, and a biologically effective amount of a vasoconstrictor selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

9. A device for transdermally delivering a biologically active agent and a vasoconstrictor, comprising:
a member having a plurality of stratum corneum-piercing microprotrusions; and
a dry coating disposed on at least one of said plurality of stratum corneum-piercing microprotrusions, said coating including a biologically active agent and a vasoconstrictor.

10. The device of claim 9, wherein said biologically active agent comprises a vaccine selected from the group consisting of conventional vaccines, recombinant protein vaccines, DNA vaccines and therapeutic cancer vaccines.

11. The device of claim 9, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserlin, triptorelin, interferon alpha, interferon beta, interferon gamma, FSH, EPO, GM-CSF, G-CSF, IL-10, glucagon, growth hormone releasing factor (GRF) and analogs of these agents, including pharmaceutically acceptable salts thereof and mixtures thereof.

12. The device of claim 9, wherein said vasoconstrictor is selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

13. The device of claim 12, wherein said vasoconstrictor comprises in the range of 0.1-10.0 wt. % of said coating.

14. The device of claim 9, wherein each of said plurality of stratum corneum-piercing microprotrusions has a length less than approximately 1000 microns.

15. The device of claim 14, wherein each of said plurality of stratum corneum-piercing microprotrusions has a length less than approximately 500 microns.

16. The device of claim 9, wherein each of said plurality of stratum corneum-piercing microprotrusions has a thickness in the range of approximately 5-50 microns.

17. The device of claim 9, wherein said coating has a thickness less than 50 microns.

18. The device of claim 17, wherein said coating thickness is less than 10 microns.

19. The device of claim 9, wherein each of said plurality of stratum corneum-piercing microprotrusions includes in the range of 1 microgram to 1 milligram of said biologically active agent.

20. A device for transdermally delivering a biologically active agent and a vasoconstrictor, comprising:
a member having a plurality of stratum corneum-piercing microprotrusions, each of said microprotrusions having a length of less than 1000 microns and a thickness less than 50 microns; and
a dry coating disposed on said member, said coating including a biologically active agent and a vasoconstrictor.

21. A method of making a device for transdermally delivering a biologically active agent and a vasoconstrictor, the method comprising:
providing a member having a plurality of stratum corneum-piercing microprotrusions, said microprotrusions having a length of less than 1000 microns;
applying an aqueous solution of a biologically active agent and a vasoconstrictor onto the member; and
drying said applied aqueous solution to form a dry agent-containing coating on said member.

22. The method of claim 21, wherein said biologically active agent comprises a vaccine selected from the group consisting of conventional vaccines, recombinant protein vaccines, DNA vaccines and therapeutic cancer vaccines.

23. The method of claim 21, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserlin, triptorelin, interferon alpha, interferon beta, interferon gamma, FSH, EPO, GM-CSF, G-CSF, L-10, glucagon, growth hormone releasing factor (GRF) and analogs of these agents, and mixtures thereof.

24. The method of claim 21, wherein said vasoconstrictor is selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

25. The device of claim 24, wherein said vasoconstrictor comprises in the range of 0.1-10.0 wt. % of said coating.

26. The method of claim 21, wherein said coating is applied by dip coating.

27. The method of claim 21, wherein said coating is applied by spray coating.

28. The method of claim 21, wherein said coating is applied by pattern coating.

29. A method of making a device for transdermally delivering a biologically active agent and a vasoconstrictor, the method comprising:
providing a sheet member;
etching a microprojection array on said sheet member to form a plurality of microprojections;
bending said plurality of microprojections whereby said plurality of microprojections project from a plane of said sheet member;
coating at least a first microprojection of said plurality of microprojections with an aqueous solution containing a biological active agent and a vasoconstrictor; and
drying said applied aqueous solution to form a dry agent containing coating on said first microprojection.

30. The method of claim 29, wherein each of said plurality of microprojections are coated with said aqueous solution.

31. The method of claim 29, wherein each of said plurality of microprojections has a length less than 1000 microns.

32. The method of claim 29, wherein each of said plurality of microprojections are bent at an angle of approximately 90° relative to said sheet member plane.

33. The device of claim 29, wherein said biologically active agent comprises a vaccine selected from the group consisting of conventional vaccines, recombinant protein vaccines, DNA vaccines and therapeutic cancer vaccines.

34. The method of claim 29, wherein said biologically active agent is selected from the group consisting of ACTH (1-24), calcitonin, desmopressin, LHRH, LHRH analogs, goserelin, leuprolide, parathyroid hormone (PTH), vasopressin, deamino [Val4, D-Arg8] arginine vasopressin, buserlin, triptorelin, interferon alpha, interferon beta, interferon gamma, FSH, EPO, GM-CSF, G-CSF, IL-10, glucagon, growth hormone releasing factor (GRF) and analogs of these agents, and mixtures thereof.

35. The method of claim 29, wherein said vasoconstrictor is selected from the group consisting of amidephrine, cafaminol, cyclopentamine, deoxyepinephrine, epinephrine, felypressin, indanazoline, metizoline, midodrine, naphazoline, nordefrin, octodrine, orinpressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and mixtures thereof.

36. The device of claim 35, wherein said vasoconstrictor comprises in the range of 0.1-10.0 wt. % of said coating.

37. The method of claim 29, wherein said coating is applied by dip coating.

38. The method of claim 29, wherein said coating is applied by spray coating.

39. The method of claim 29, wherein said sheet member is formed from a material selected from the group consisting of stainless steel and titanium.
